(19) United States
(12) Patent Application Publication
(10) Pub. No.: US 2012/0130060 A1
(43) Pub. Date: May 24, 2012

(54) Modified antisense oligopeptides with anticancer properties and method of obtaining them

(76) Inventors: Artur Martynov, Kharkov (UA); Boris S. Farber, Brooklyn, NY (US); Sonya Sophya Farber, New York, NY (US)

(21) Appl. No.: 12/931,468

(22) Filed: Feb. 1, 2011

Related U.S. Application Data
(63) Continuation of application No. PCT/RU2010/000691, filed on Nov. 22, 2010.

(51) Int. Cl. C07H 21/00; C07H 1/00; C12P 19/34

(52) U.S. Cl. 536/24.5; 435/91.53; 536/25.3

(57) ABSTRACT

This invention may be used in human and veterinary medicine for the creation of a drug that is effective in the treatment of oncological illnesses in animals and humans.

Summary of the Invention

Modified antisense oligonucleotides with anticancer properties and methods of obtaining them, distinct in that in the capacity of oligonucleotides, a mixture of the products of the hydrolysis of polynucleotides is used, and modification is done through changing the molecular charges of the nucleotide bases to their opposites; they thereby obtain antisense properties. The hydrolysis of polynucleotides leads to the application of natural or synthetic nucleases, acid or alkaline hydrolysis, and modification of the structure through the acylation of the amino groups of mononucleotides in the structure of oligonucleotides by dicarboxylic acids or through their alkylation by halogen-carboxylic acids. The mixture developed is capable of selectively bonding with microRNA, thereby stopping the synthesis of protein in cancer cells that is similar to microRNA activity.

The application of this drug in connection with its ability to adapt to the body allows the elimination of a tumor's tolerance to the drug. This drug has a wide spectrum of activity and a low level of toxicity; it is accessible for industrial production and effective at any stage of cancer.

### TECHNICAL FIELD

This invention is related to medicine—specifically, to pharmacology and oncology—and is intended for the treatment of oncologic diseases in humans.

### PREVIOUS LEVEL OF TECHNOLOGY

Among the existing new fields in the treatment of oncological diseases, there are several quite promising approaches. One of these approaches may be considered the development of drugs for cancer gene therapy [¹]. In this approach, the main active principle is polynucleotides. Gene therapy can be divided into two groups: means to inactivate genes [²] and means for introducing genetic material into a cell [³]. Gene inactivators are also called antisense polynucleotides [⁴]. Many research projects conducted in this direction have not come up with truly effective in vivo drugs. This is connected with a whole host of problems: the synthesized DNA (RNA) was quickly destroyed by blood nucleases [⁵], did not penetrate the cells, and the genome repair systems were disrupted [⁶]. Sometimes, a short-term block of the expression of protein targets and buildup in the hepatocytes has been observed [⁷]. There are various approaches to the design of oligonucleotide antisense, but the principle of their interaction with the target remains the same: the creation of hydrogen bonds between complementary nucleotides upon increasing the level of resistance to nuclease [⁸]. In certain cases, developers have protected the 5′ end of an oligonucleotide from nuclease action; in other cases, they have modified the 3′ end [⁹,¹⁰]. Researchers from the US have exchanged deoxyribosil remnants for fragments of morpholine with the goal of creating fragments of DNA (RNA) that can withstand nuclease action [¹¹, ¹²]. The principle of gene inactivation through their complementary interaction with antisense nucleotides has remained the same: the creation of hydrogen bonds. It is this hydrogen bond that is the main reason for the ineffectiveness of existing drugs based on antisense DNA (RNA). Ferments of a type of helicase [¹³] very quickly and easily unwind antisense DNA that is hybridized with the gene target, and the gene's activity begins again.

We know of nucleotide sequences that are connected with the development of prostate and lung cancer [¹⁴]. The mechanism of activity of this microRNA is connected with the suppression of protein synthesis through blocking the translation of messenger RNA. The authors have patented microRNA sequences, confirmed their effects on cell cultures, and demonstrated the specificity of these nucleotides to these two types of tumors. The main shortcomings of the patent are that the authors did not demonstrate possible methods of application of the given microRNA for the battle against oncological illnesses; they did not demonstrate how to protect these oligonucleotides against the activity of nuclease in the body, and they did not demonstrate the anti-cancerous effect of the drugs based on this microRNA.

The most similar prototypes are the oligonucleotides containing modified and synthetic nucleotide bases [¹⁵]. The mechanism of action of the patented oligonucleotides was analogous to antisense RNA and microRNA, and the drugs could be used for treatment of oncological diseases, among other things. A shortcoming of the oligonucleotides being patented is the application of the natural principle of the creation of bonds with RNA targets; this is a hydrogen bond, which is sensitive to nucleases and helicases. In addition, a precise static chemical structure has been patented that is not capable of adapting to surrounding conditions. Application of the object of this patent was not effective in experiments on models of animals with oncological diseases; moreover, this development is applicable more for in vitro diagnostic and screening studies than for the creation of drugs for the treatment of animals and humans in connection with the fact that the proposed modifications to the nucleotide bases are new xenobiotics and are not subject to safe metabolism and biodegradation in the body.

### DISCLOSURE OF THE INVENTION

The task of the invention was to develop modified antisense oligonucleotides with anti-cancer properties and methods of obtaining them.

The task set is addressed through obtaining modified antisense oligonucleotides with anticancer properties and methods of obtaining them, distinct in that in the capacity of oligonucleotides, a mixture (assembly) of the products of the hydrolysis of polynucleotides is used, and modification is done through changing the molecular charges of the nucleotide bases to their opposites; they thereby obtain antisense properties.

The task set is also addressed through the development of a method of obtaining modified antisense oligonucleotides with anticancer properties, distinct in that to obtain them, first a partial hydrolysis of polynucleotides (DNA, RNA, or a combination of the two) of plant, animal, microbial, or fungal is conducted through chemical or biochemical (enzymatic) procedures, and then a process of chemical modification of the oligonucleotides obtained with a change in the molecular charge of their nucleotide bases to the opposite is conducted through alkylation with monochloracetic acid or acylation with succinic anhydride; a mixture (assembly) of the antisense oligonucleotides obtained is used in the capacity of an anticancer drug.

### SHORT DESCRIPTION OF DRAWINGS

FIG. 1a. Specific Hybridization Of Acylated DNA (RNA) Only With Their Precursors

FIG. 1b. Specific Hybridization Of Acylated DNA Plasmids Only With Their Precursors

FIG. 2. Replacement of a hydrogen bond with an ionic bond in the creation of hybrids between Anticanum and the target

FIG. 3. Sarcoma. Hematoxylin-Eosin Dye

### BEST INVENTION IMPLEMENTATION OPTION

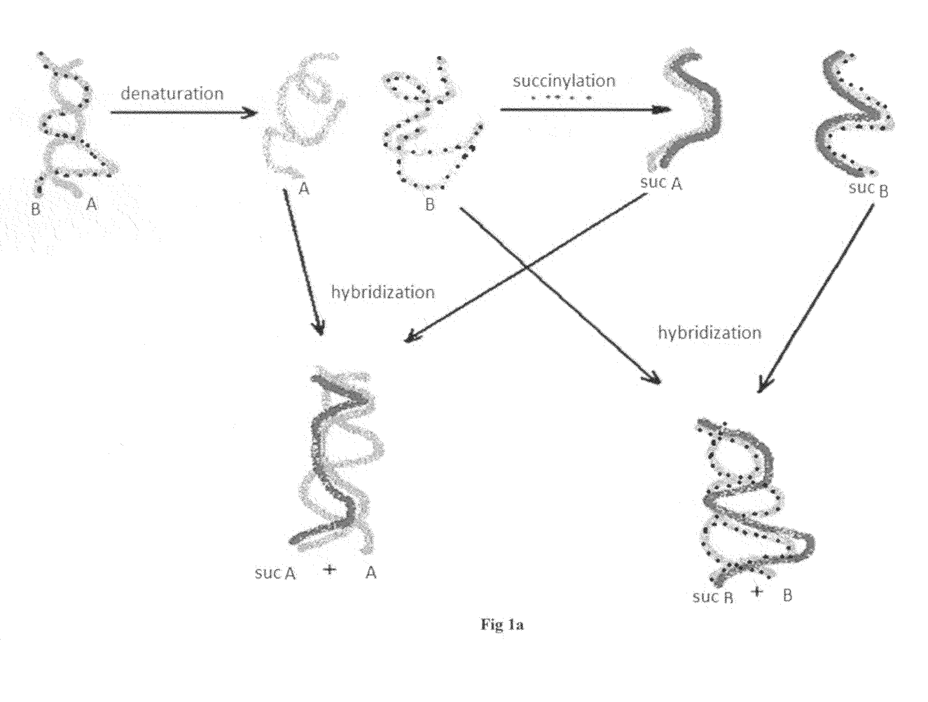

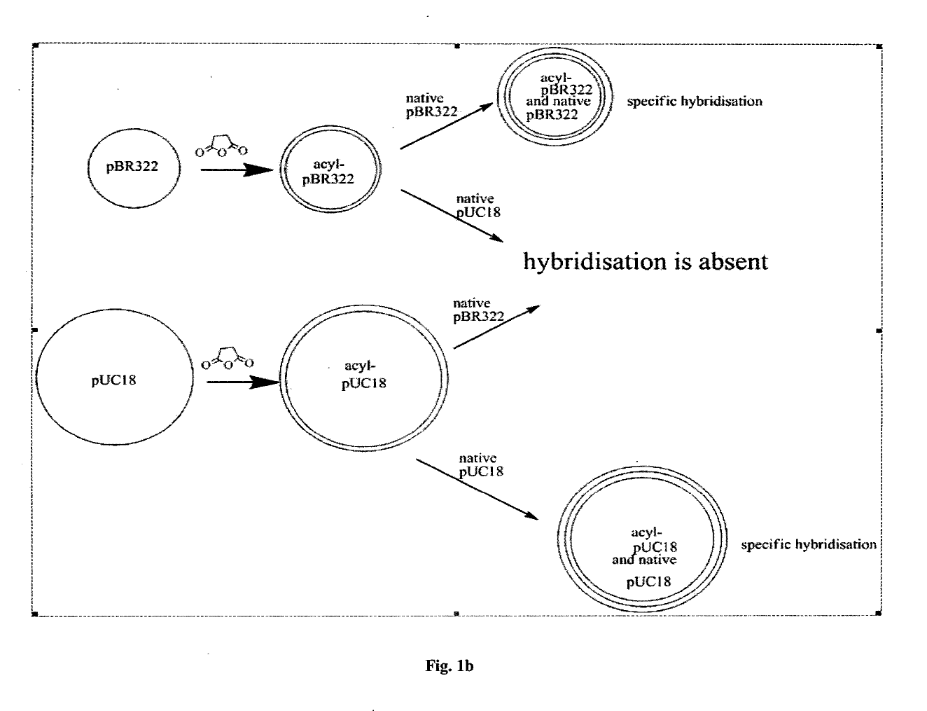

In our earlier studies, when researching acylated polynucleotides, we discovered a new phenomenon: polynucleotides that had been acylated along exocyclic amino groups hybridize selectively only with their own non-acylated precursors. The pUC18 plasmid hybridized only with its acylated derivatives, while the pBR322 plasmid hybridized, accordingly, only with acylated pBR322 (FIG. 1b). Insoluble, non-melting conjugates were created. It was the non-melting property that distinguished the classic, double-helix DNA from the hybrids that had been obtained. They would not melt at any temperature and would not dissolve in anything except in concentrated alkalis. We explain this property of the synthesized acyl-DNA by a change to the very principle of bonding between DNA chains: from hydrogen to ionic and mixed.

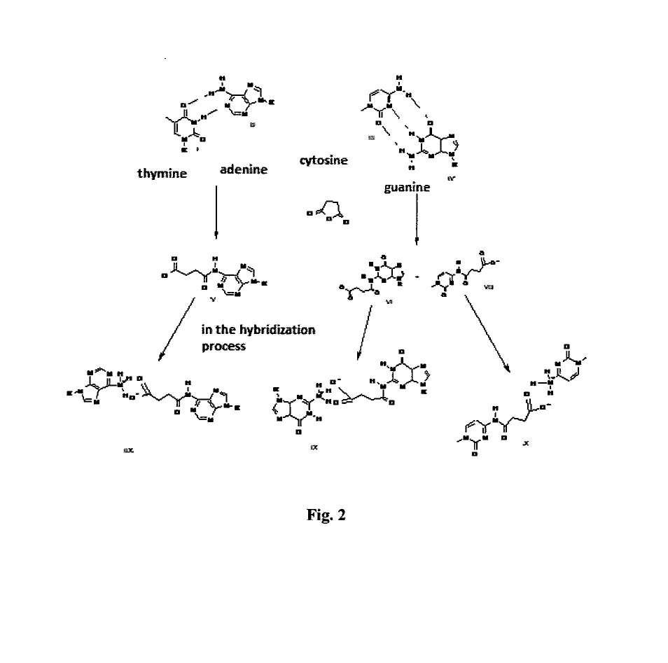

This bond (FIG. 2) was not stipulated at all by the natural repair mechanisms. Thus the cell helicases and nucleases will be ineffective if hybrids are created between this type of acyl-DNA (RNA) and its non-acylated predecessors. In addition to the polynucleotide phenomenon, we also saw a dependence between charge and activity in a series of other acylated biopolymers: proteins, polysaccharides, polynucleotides, tannins, bacteriophages, and immunoglobulins. On the basis of this research, a new veterinary antiviral drug was developed and implemented [¹⁶]. We established that a certain precision modification to the structure of a biopolymer is capable of increasing its biological activity, fully changing its properties, or leading to the creation of self-organizing structures. The behaviors we discovered were taken as the basis for a principle of obtaining microRNA from acylated exocyclic amino groups. We used an assembly of oligopeptides that were the product of the autological hydrolysis of polynucleotides, but the molecules' charges were changed to the opposite. “Assembly” is a term from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell [¹⁷,¹⁸]

This drug was named Anticanum. Earlier, we discovered the liposomal forms of certain promising drugs; the medicinal form for Anticanum was developed on the basis of these data.

This microRNA is a new class of uncoded RNA with a length of 18-25 nucleotides, which negatively regulate post-translation gene expression. The mechanism of the activity of these small fragments of RNA is based on the interaction (hybridization) of microRNA with matrix RNA directly in the polyribosome complex. This hybridization will lead to the cessation of the synthesis of a specific protein. The role of microRNA in carcinogenesis and perspectives on using microRNA in cancer therapy is presented in a review [¹⁹]. However, microRNA is not capable of penetrating a cell membrane on its own. Currently, research is being done on the creation of various transportation systems for the delivery of microRNA to cell targets. The most promising carriers are liposomes, polymer nanoparticles, and self-organizing polymers.

The ability of many adenocarcinomas to pick up oligonucleotides and nanoparticles by pinocytosis from the intercellular matrix is known [²⁰,²¹]. Healthy cells are not capable of picking up small oligonucleotides and liposomes [²²]. This facilitates Anticanum aggregation selectivity in cancerous cells and the drug's lack of toxicity. In order to obtain Anticanum, we used accumulated RNA from yeast, fragmented pancreatic nuclease to fragments from 2-15 n in size. Then the exocyclic amino groups of these oligonucleotides were modified by changing their charges. For purposes of improving Anticanum's aggregation in tumors, it was included in monolamellar phosphatidylcholine liposomes. The selective aggregation of Anticanum in cancer cells leads to its hybridization with complementary targets in the matrix RNA of the cancer cell and to the gradual cessation of protein synthesis. Anticanum blocks not only the intronic, but also the exonic areas of matrix RNA, which leads to the practically instantaneous stoppage of protein synthesis. Anticanum's activity is based on the inducement of apoptosis through the stoppage of protein synthesis. Anticanum does not affect cellular health, blocks the synthesis of all cellular proteins, and excludes the adaptation of the cancer tumor to therapy and the selection of resistant cells.

The molecular mechanism of the drug's activity has not been studied.

In addition to showing anticancer activities in vitro, Anticanum also showed a high activity in vivo on models of benzidine sarcomas in rats and Ehrlich's ascites adenocarcinoma in mice (presented later in the report).

### Example 1

### Obtaining an Assembly (Mixture) of Modified Oligonucleotides (Anticanum)

The microbial biomass contains up to 11% nucleic acids and may serve as a raw material for the obtainment of microbial RNA, on the basis of which it is possible to obtain derinates that are a mixture of oligonucleotides. The object of study for this stage of the work was RNA taken from Saccharomyces cerevisiae yeast biomass.

Separation of Summary RNA from Baker's Yeast. Four kg of compressed yeast was thawed at room temperature. It was ground and suspended in 8 l of boiling water containing 300 g of sodium dodecal sulfate. The suspension was boiled for 40 minutes while being stirred constantly. It was then poured into steel centrifuge cups, which were quickly placed in ice and cooled to 5° C. (˜15 min.), after which they were centrifuged in a 6K15 German-made centrifuge (17000 g, 10 min, 4° C.). The sediment and a part of the gel-like interphase were removed, and the RNA that had migrated to the supernatant was separated out. This was accomplished by adding NaCl to the supernatant obtained in the previous stage, until a final concentration of 3 M was reached. After dissolving the salt, the suspension was left for 1 hour to allow the formation of sediment, which was then separated by a centrifuge at 17000 g over 10 min. The sediment was rinsed with two portions of 8 l 3 M NaCl each, suspended in 2 l of ethanol, and left overnight. The next day, the suspension was centrifuged at 17000 g over the course of 10 minutes. The sediment was dissolved in distilled water to an RNA concentration of 450 D260 U/ml (˜1.6 l). The solution was clarified by centrifuging under the same conditions, after which the RNA was precipitated from the supernatant through adding NaCl to a final concentration of 0.15 M and an equal volume of ethanol (˜2 l). The sediment formed was separated from the supernatant through centrifuging (17000 g, 10 min), cleaned in 1 l of ethanol, and dehydrated in a CaCl vacuum desiccator. All operations were conducted at a temperature of 0-4° C.

Separation of High-Polymer RNA from Baker's Yeast

Day 1: RNA Extraction.

7.5 g of sodium dodecal sulfate were dissolved in 300 ml of water in a heat-resistant one-liter glass beaker. The solution was brought to a boil, and over a period of 5 minutes, 30 g of dry yeast were added in such a manner that the temperature of the suspension did not fall below 98° C. The suspension obtained was boiled for 40 minutes while being stirred constantly, and boiling water was added to keep the mixture at 300 ml as the original water boiled off. At the end of the extraction, the suspension was cooled to approximately 60° C. Freshly boiled water was added to a volume of 450 ml. This was mixed, poured into a 500-ml measuring cylinder, and left to settle at a temperature of 20° C. for 22 hours.

Day 2: RNA Desalting and Rinsing the Desalted RNA with 3 M NaCl.

The cooled supernatant (280 ml) was transferred to a measured glass beaker with a volume of 500 ml. 81 g of NaCl was added to it, as was water, to make a total of 450 ml. This was mixed and left to settle at a temperature of 19° C. for 6 hours. After 6 hours, the interphase formed (250 ml) was bled off, and to the desalted RNA that was divided into two fractions (one part deposit, one part supernatant) was added 3 M NaCl up to 450 ml. This was mixed and left to settle overnight at a temperature of 19° C.

Day 3: Rinsing the Desalted RNA with 3 M NaCl.

The interphase (300 ml) was bled off, and 3 M NaCl was added to the desalted RNA up to 450 ml. This was mixed and left to settle at a temperature of 19° C. for 5 hours. After 5 hours, the interphase (280 ml) was bled off, and 3 M NaCl was added to the desalted RNA up to 450 ml. This was mixed and left to settle at a temperature of 19° C. for 3 hours. After 3 hours, the interphase (250 ml) was bled off, and 3 M NaCl was added to the desalted RNA up to 450 ml; this was mixed and left to settle overnight at a temperature of 19° C.

Day 4: Rinsing the Desalted RNA with Alcohol.

There is almost no top layer. Sediment occupies a volume of ˜100 ml. The supernatant was removed, and ethanol was added to the sediment up to a volume of 300 ml. This was mixed and left to settle at a temperature of 18° C. for five hours. After five hours, the supernatant was poured off, and a fresh portion of ethanol was added to the 140 ml of sediment up to a volume of 320 ml. This was mixed and left to settle at a temperature of 19° C. for 40 minutes, after which the supernatant was poured off. 120 ml of fresh ethanol was added to the 120 ml of sediment. This was mixed, and the supernatant was poured off after the mixture had been left to stand for 30 min. To the sediment (110 ml) was added 110 ml of fresh ethanol. This was mixed, and after 30 minutes of settling, the supernatant was poured off, and the RNA suspension was poured in a thin layer into a flat pan and air-dried at room temperature until the odor of alcohol had disappeared. The pure high-polymer RNA from the intermediate product was removed through water extraction in a cellophane dialysis bag.

Enzymatic Splitting of the Total RNA Obtained.

In the capacity of a nuclease, pancreatic ribonuclease (RNAase) with an activity of 14000 U/mg in a quantity of 0.4% of the mass of the RNA. The RNA splitting was conducted over a period of four hours. The hydrolyzate was dehydrated in a flash drier.

Chemical Modification of the Hydrolyzate's Oligonucleotides.

A 3.5% water solution of the hydrolyzate was obtained; succinic anhydride was added in a quantity of 10-45% of the dry weight of the hydrolyzate; this was mixed in the cold until the anhydride was fully dissolved. The solution obtained was sterilized for 120 with flowing steam. The prepared solution was studied further for presence of anticancer properties.

### Example 2

### A Study of Anticanum's Toxicity Levels

Thirty-six series of experiments were conducted on 220 animals.

The average lethal intravenous dose of Anticanum was established for three types of animals: non-speciated white mice weighing 20-22 g, white rats weighing 190-200 g, and guinea pigs weighing 300-400 g of both sexes. With the goal of comparing the spectrum of therapeutic activity of Anticanum and the comparison drug, Taxotere, we also established the average lethal intravenous dose of Taxotere.

The calculation of the average lethal dose was made according to B. M. Shtabskiy's methods with the use of the equation:

$$X = \frac{Y - a}{a} \qquad ( 1 )$$

Where Y—% of effect

$$a = \frac{{Y}_{1} - {Y}_{2}}{{X}_{1} - {X}_{2}}$$

$$b = \frac{\sum Y - a \sum X}{n}$$

where:
X1 and X2 are the values of the two extreme of the three doses studied that have an effect on more or less than 50% of the animals; the third dose is the intermediate;
Y1 [symbol] Y2 are the lethality percentages corresponding to doses X1 and X2;
ΣY— is the sum of the three doses studied;
   the number of doses used in the calculations is equal to 3.

If we add to formula (I) a value of Y that is 50, 84, and 16 percent mortality, LD₅₀, LD₈₄, and LD₁₆ are calculated. Then d, the average margin of error for the average lethal dose was found through use of the Miller-Tainter formula:

$$m = \frac{2 δ}{\sqrt{2 N}} - {LD}_{84} - {LD}_{16} ;$$

N is the total number of animals in the groups in which even one animal died or survived, and the confidence ranges were established.

The mice were dosed by a slow intravenous method with Anticanum (vector liposomes were prepared before use: a 0.9% solution of NaCl was added to the dry lyophilized Anticanum powder that was taken in corresponding dosages) in dosages of 1000, 1500, 2000, 2500, and 3500 mcg/kg animal body weight. An analysis of the results indicates that the average lethal dosage of the drug for this species of animal is 2780 mcg/kg.

A study of Anticanum in rats at dosages of 1000, 2000, 2500, 3000, 3500, and 4000 mcg/kg provided the opportunity to determine that the average lethal dosage for this species of animal is 2590 mcg/kg. The average lethal dosage of Anticanum for guinea pigs is 2420 mcg/kg weight.

Intravenous Taxotere in rats showed LD50=120 mcg/kg body weight.

A comparison of the characteristics of Anticanum and Taxotere is presented in Table 5.

TABLE 1
Comparative Characteristics of Anticanum and Taxotere

| | | LD50, mcg/kg in | | Relative |
|---|---|---|---|---|
| | ED50, | Rats, Intravenous | Therapeutic | Therapeutic |
| Drug | mcg/kg | Introduction | Index | Index |
| Anticanum | 5 | 2590 | 518 | 151.5 |
| Taxotere | 35 | 120 | 3.42 | 1 |

An analysis of the data obtained, which are presented in Table 1, leads to the conclusion that Anticanum in liposomes is less toxic than the drug Taxotere, and exceeds that drug in comparison of effective dosage. The therapeutic index that characterizes the breadth of therapeutic activity was 151.5 times more for Anticanum than in the comparison drug due to the liposomal form. In order to extrapolate Anticanum's toxicity in humans, we calculated a species sensitivity ratio (SSR) according to the formula:

$$SSR = \frac{LD 50 ( for rats )}{LD 50 ( for mice )} = \frac{2590}{2780} = 0.9$$

Thus Anticanum does not have a species-related sensitivity, or it appears slowly.

### Example 3

### The Anticancer Activity of Anticanum

The determination of the anticancer activity of Anticanum in a cell culture was made in a culture of HeLa-2 cells. For this purpose, 2-12 mcg of Anticanum per ml of medium were added to the 199 medium. A culture without Anticanum in it was used as a control. Cultures were observed daily over the course of five days. The Minimum Active Dose (MAD) of Anticanum was also considered to be the minimum amount of the drug that caused a degeneration of 90-95% of the cells (Table 2.

TABLE 2
Comparative Sensitivity Characteristics of Cultures of
HeLa-2 Tumor Cells to Anticanum

| | | Anticanum Activity at Various Acidity | |
|---|---|---|---|
| | MAD in | Levels among 199 ¹ Cell Cultures. | |
| Drug | mcg/ml | Control | Experiment |
| Anti- | 10 | 0 | ++++ |
| canum | | | |
| Taxotere | 10 | 0 | ++ |
| Bare lipo- | — | 0 | 0 |
| somes | | | |

¹ Cytopathic activity;
++++ degeneration of 100% of the cells
0 lack of degeneration.

In establishing the minimum concentration of Anticanum that will slow the growth of cells, a comparison was made between the number of surviving cells and the concentration of Anticanum in the solution.

TABLE 3
The Effect of Anticanum on HeLa Cells

| | | Number of Live | | Number of | |
|---|---|---|---|---|---|
| | | Cells after | | Live Cells | |
| | | Incubation, | | after Incubation, | |
| | Number of cells | Millions, ±1000 | | Millions, % | |
| | before Incubation, | Anti- | | Anti- | |
| Dose, mcg/ml | Millions | canum | Taxotere | canum | Taxotere |
| 2 | 150000 ± 1000 | 72000 | 150000 | 48 | 100 |
| 4 | 153000 ± 1000 | 21400 | 150000 | 14 | 98 |
| 6 | 150000 ± 1200 | 9800 | 145000 | 6.5 | 97 |
| 8 | 152000 ± 1000 | 0 | 135000 | 0 | 89 |
| 10 | 158000 ± 1000 | 0 | 130000 | 0 | 82 |
| 12 | 162000 ± 1000 | 0 | 153000 | 0 | 94 |

As may be seen in Table 3, an effective dose of Anticanum is between 8-12 mcg/ml solution.

Anticanum led to a 95% degeneration of tumor cells. To confirm the in vivo antitumor activity, Anticanum was studied in benzidine skin sarcoma and reinjected ascites adenocarcinomas in Barbados mice. In five mice with adenocarcinomas, the distribution of the Anticanum liposome throughout the animals' bodies was also studied using a fluorescent probe dissolved in a phospholipid layer.

### Example 4

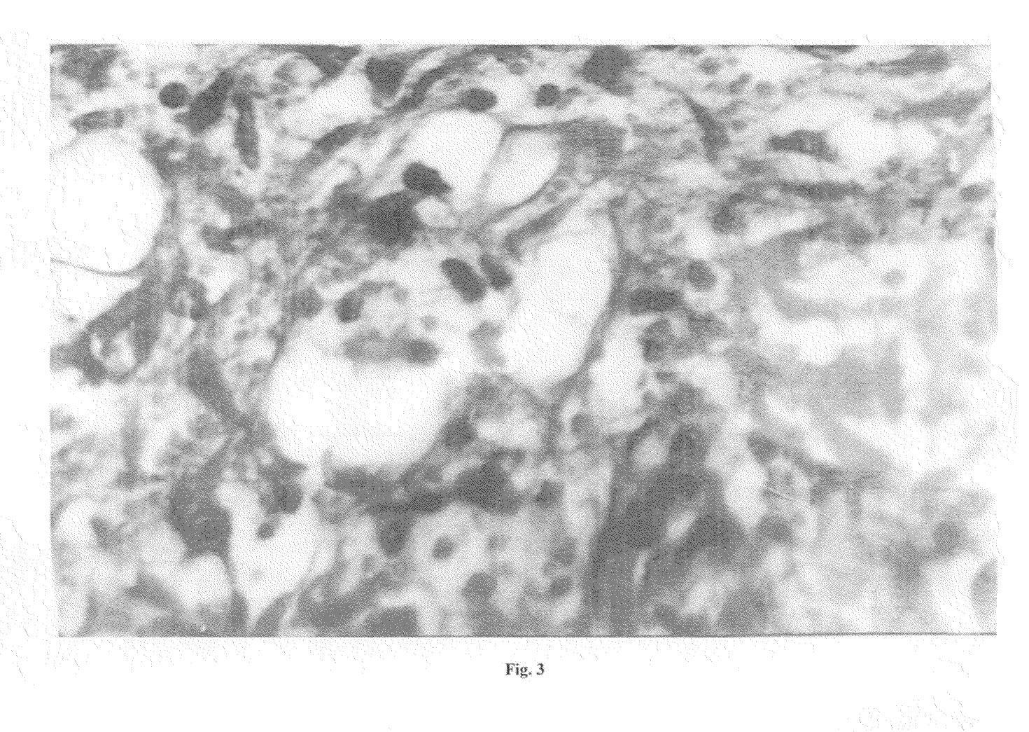

### A Study of the Anticancer Activity of Anticanum on Benzidine Sarcoma (FIG. 3.)

Before applying it to the silica gel, 7 ml of a solution of 2% benzidine and 0.9% sodium chloride was added until an opalescent suspension was formed (1 g silica gel for 5 ml NaCl solution). Twenty-five Barbados mice of both sexes with a weight of 18-20 g that were kept on a vivarium diet were administered benzidine and phorbol acetate immobilized on silica gel subcutaneously near the neck. After two weeks, 18 animals had developed tumors of different sizes in the form of a small bump on the neck near the silica gel granulomas. Each group of animals was administered the corresponding compound parenterally at a dose of 100 mcg/kg weight twice a day for two weeks, beginning at 16 days after administration of the carcinogen.

TABLE 4
A Comparison of the Antitumor Activity of Anticanum in Comparison
to the Combination of an Analog (Taxotere) and a Lipid

| | Weight of Animal (g) | |
|---|---|---|
| Drug Name | Before Treatment | After Treatment |
| Taxotere | 28 ± 1.2 | 23 ± 1.1 (2 mice died) |
| Anticanum | 25 ± 1.7 | 15 ± 1.5 |
| Bare Liposomes | 26 ± 2.1 | 34 ± 1.3 (5 mice died) |

Note:
n = 7, p > 0.05 in comparison with the control and previous data.

As may be seen in Table 4, Anticanum decreased the weight of the experimental animals by 10 g; the control animals' weight continued to increase, and some of them died. After the dissection of the silica gel granulomas, it was established that the animals treated with Anticanum did not show signs that the granulomas had turned into malignant sarcomas.

The animals' survival rates are presented in Table 5.

TABLE 5
Survival Rates of Animals with Benzidine Skin Sarcoma

| Drug Name | Animal Survival, Days |
|---|---|
| Taxotere | 28 ± 1.1 |
| Anticanum | 49 ± 1.2 |
| Bare Liposomes | 17 ± 0.9 |

Note:
n = 10, p > 0.05 in comparison with the control and previous data.

Thus Anticanum prolongs the life of animals twice as long as does Taxotere.

### Example 5

### A Study of the Antitumor Activity of Anticanum on Ehrlich's Ascites Adenocarcinoma

The antitumor activity of the compositions were studied in models of Ehrlich's ascites carcinoma in young Barbados mice of both sexes with weights between 15-17 g (68 individuals), which were kept on a vivarium diet.

45 mice were inoculated from a mouse with adenocarcinoma using an insulin syringe with 0.1 ml ascitic fluid in the region of the liver. Within seven days, 42 mice showed signs of tumors (the body weight and belly size increased); two mice died on the second day; one mouse did not show signs of a tumor.

Ten mice were administered Anticanum in liposome form (see Table 6). Ten more mice were administered the Anticanum substance, and ten more were administered a 0.9% solution of sodium chloride with lipids.

TABLE 6
Qualitative Biological and Statistical Characteristics in the Study
of Anticanum's Antitumor Activity

| | | Time of Death | |
|---|---|---|---|
| | | of Animals after the First Injection | |
| | | Average Value | |
| | | Days | |
| Substance | Liposomes | Experimental Animals | Control Animals |
| Anticanum | + | 37.4 ± 0.88 | 3.2 ± 0.44 |
| −//− | − | 18 ± 3.2 | 3.1 ± 0.48 |
| Taxotere | + | 15 ± 0.5 | 3 ± 0.5 |
| −//− | − | 14 ± 0.12 | 3 ± 0.6 |

Note:
n = 10, p > 0.05 in comparison with the control and previous data.

Anticanum was given to those mice from which blood was drawn. Mice with Ehrlich's adenocarcinoma, after being given the tumor and treated, lived for 18 days when administered the Anticanum substance, which is 6 times longer than the control, and 37 days when administered Anticanum in liposomes, which is 12 times longer than the control. At an accuracy level of more than 99.5%, we can confirm a significant increase in anticancer activity in liposomal Anticanum over the control, Taxotere. After dissection of the animals, signs of tumors and metastasis were not found in their bodies.

### Example 6

### Study of the Distribution of the Anticanum Liposome Throughout the Bodies of Animals

For the study of the distribution of the liposome throughout animals' organs, biologically inert 5,6-benzocoumarin was used, which is lipophilic and has fluorescent properties. The benzocoumarin was diluted in alcohol (0.5 M solution) and added to a phospholipid solution upon creation of the liposome in the quantity of 0.01 ml coumarin solution in 10 ml of the phospholipid solution. The most fluorescence was observed in the liver and ascitic fluids, which confirms the liposome's affinity for the tumor. In the control, the liposomes were distributed in the spleen, liver, and lymph nodes.

The distribution of the liposomes was studied in the bodies of mice with tumors (five animals) and healthy rats (five animals) after a fluid administration of Anticanum. Anticanum was administered at a dosage of 100 mcg/kg. After five hours, the mice were pithed and the fluorescence intensity was studied using an HP-F-40M fluorometer.

TABLE 7
Accumulation of Anticanum Dependent on Speed of Introduction

| | | Form of Anticanum | |
|---|---|---|---|
| Animals | Organ | Introduction | Fluorescence, % * |
| healthy rats | | slow infusion | |
| −∥− | plasma | | 23.4 ± 0.1 |
| −∥− | liver | | 26.1 ± 0.1 |
| −∥− | lungs | | 0.5 ± 0.1 |
| −∥− | spleen | | 6.2 ± 0.1 |
| −∥− | kidneys | | 32.2 ± 0.1 |
| −∥− | blood | | 25.6 ± 0.1 |
| | fast intravenous introduction | | |
| −∥− | plasma | | 25.3 ± 0.1 |
| −∥− | liver | | 56.2 ± 0.1 |
| −∥− | lungs | | 0.5 ± 0.1 |
| −∥− | spleen | | 0.3 ± 0.1 |
| −∥− | kidneys | | 0.2 ± 0.1 |
| −∥− | blood | | 5.5 ± 0.1 |
| mice with | slow infusion | | |
| adenocarcinoma | | | |
| −∥− | plasma | | 15.2 ± 0.1 |
| −∥− | liver | | 12.2 ± 0.1 |
| −∥− | lungs | | 0.5 ± 0.1 |
| −∥− | spleen | | 8.2 ± 0.1 |
| −∥− | kidneys | | 52.2 ± 0.1 |
| −∥− | ascitic fluid | | 45.6 ± 0.1 |
| | fast intravenous introduction | | |
| −∥− | plasma | | 23.2 ± 0.1 |
| −∥− | liver | | 60.2 ± 0.1 |
| −∥− | lungs | | 0.2 ± 0.1 |
| −∥− | spleen | | 0.5 ± 0.1 |
| −∥− | kidneys | | 0.3 ± 0.1 |
| −∥− | ascitic fluid | | 5.2 ± 0.1 |

* relative fluorescence: the percentage of fluorescence relative to the intensity of the fluorescence of the administered Anticanum solution.

As can be seen in the table, when a tumor is present, the aggregation of Anticanum goes there, but in dependence on the time taken for the infusion. The faster Anticanum is introduced, the more of it is collected in the liver. It is therefore rational to introduce Anticanum via slow infusion.

This invention is related to human and veterinary medicine—specifically to oncology—and may be used in the treatment of oncological infections in animals and humans.

### INDUSTRIAL APPLICABILITY

This invention is related to medicine and pharmaceuticals—specifically to oncology and pharmaceuticals—and may be used for the design and creation of new, more effective drugs that will be self-adapting and self-organizing systems. The drugs obtained in this manner are completely ecologically safe, biodegradable, and fully metabolized both in patients' bodies and in the environment; the technology required to make them is completely without waste. The production of the product being patented can be done on the existing equipment of pharmaceutical companies and does not require unique equipment. The raw base is accessible and does not require additional efforts for growth or production.

### REFERENCES

1 Galasso M, Elena Sana M, Volinia S, Non-coding RNAs: a key to future personalized molecular therapy?// Genome Med. 2010 Feb. 18; 2(2):12.
2 Jung J, Solanki A, Memoli K A, Kamei K, Kim H, Drahl M A, Williams L J, Tseng H R, Lee K. Selective inhibition of human brain tumor cells through multifunctional quantum-dot-based siRNA delivery// Angew Chem Int Ed Engl. 2010; 49(1):103-7.
3 Li X, Liu Y, Wen Z, Li C, Lu H, Tian M, Jin K, Sun L, Gao P, Yang E, Xu X, Kan S, Wang Z, Wang Y, Jin N. Potent anti-tumor effects of a dual specific oncolytic adenovirus expressing apoptin in vitro and in vivo// Mol Cancer. 2010 Jan. 20; 9:10.
4 Emmrich S, Wang W, John K, Li W, Pützer B M. Antisense gapmers selectively suppress individual oncogenic p73 splice isoforms and inhibit tumor growth in vivo// Mol Cancer. 2009 Aug. 11; 8:61.
5 Fisher M, Abramov M, Van Aerschot A, Rozenski J, Dixit V, Juliano R L, Herdewijn P. Biological effects of hexitol and altritol-modified siRNAs targeting B-Raf// Eur J Pharmacol. 2009 Mar. 15; 606(1-3):38-44
6 Czauderna F, Fechtner M, Dames S, Aygün H, Klippel A, Pronk G J, Giese K, Kaufmann J. Structural variations and stabilising modifications of synthetic siRNAs in mammalian cells// Nucleic Acids Res. 2003 Jun. 1; 31(11):2705-16.
7 Crooke R M, Graham M J, Martin M J, Lemonidis K M, Wyrzykiewiecz T, Cummins L L. Metabolism of antisense oligonucleotides in rat liver homogenates. J Pharmacol Exp Ther. 2000 January; 292(1): 140-9.
8 Monia B P, Johnston J F, Sasmor H, Cummins L L. Nuclease resistance and antisense activity of modified oligonucleotides targeted to Ha-ras. J Biol Chem. 1996 Jun. 14; 271(24):14533-40.
9 Giles R V, Spiller D G, Green J A, Clark R E, Tidd D M. Optimization of antisense oligodeoxynucleotide structure for targeting bcr-abl mRNA. Blood. 1995 Jul. 15; 86(2):744-54.
10 Xodo L, Alunni-Fabbroni M, Manzini G, Quadrifoglio F. Pyrimidine phosphorothioate oligonucleotides form triple-stranded helices and promote transcription inhibition. Nucleic Acids Res. 1994 Aug. 25; 22(16):3322-30.
11 Sazani P, Weller D L, Shrewsbury S B. Safety pharmacology and genotoxicity evaluation of AVI-4658. Int J Toxicol. 2010 March-April; 29(2):143-56.
12 Mourich D V, Jendrzejewski J L, Marshall N B, Hinrichs D J, Iversen P L, Brand R M. Antisense targeting of cFLIP sensitizes activated T cells to undergo apoptosis and desensitizes responses to contact dermatitis. J Invest Dermatol. 2009 August; 129(8):1945-53.
13 Belon C A, Frick D N. Helicase inhibitors as specifically targeted antiviral therapy for hepatitis C. Future Virol. 2009 May 1; 4(3):277-293
14 U.S. Pat. No. 7,709,616, MicroRNA and its uses.
15 U.S. Pat. No. 7,579,451 Oligonucleotides comprising a modified or non-natural nucleobase
16 http://www.agrovet.com.ua/index.php?id=25
17 http://dic.academic.ru/dic.nsf/ruwiki/79240
18 Jean-Marie Lehn. Supramolecular Chemistry. Concepts and Perspectives. —Weinheim; New York; Basel; Cambridge; Tokyo: VCH Verlagsgesellschaft mbH, 1995. —P. 103 (Chapter 7)
19 Bagnyukoval T. V., Pogribny I. P. Chekhun V. F. MicroRNAs in normal and cancer cells: A New class of gene expression regulators// Experimental Oncology. —2006, N 28. —P. 263-269
20 Steinhauser I, Langer K, Strebhardt K, Spänkuch B. Uptake of plasmid-loaded nanoparticles in breast cancer cells and effect on Plk1 expression. J Drug Target. 2009 September; 17(8):627-37.
21 Lu J J, Langer R, Chen J. A novel mechanism is involved in cationic lipid-mediated functional siRNA delivery. Mol Pharm. 2009 May-June; 6(3):763-71.
22 Moreira J N, Santos A, Moura V, Pedroso de Lima M C, Simões S. Non-viral lipid-based nanoparticles for targeted cancer systemic gene silencing. J Nanosci Nanotechnol. 2008 May; 8(5):2187-204.

1. Modified antisense oligonucleotides with anti-carcinogenic properties and methods of obtaining them, distinct in that in the capacity of oligonucleotides, a mixture (assembly) of the products of the hydrolysis of polynucleotides is used, and modification is done through changing the molecular charges of the nucleotide bases to their opposites; they thereby obtain antisense properties.

2. Modified antisense oligonucleotides with anticancer properties according to claim 1, distinct in that the molecular charge of the nucleotide bases is changed to the opposite through the formation of an amino group as a result of an acylation reaction between anhydrides of di- and tricarboxylic acids and the amino groups of the nucleotide bases with the creation of new carboxyl groups.

3. Modified antisense oligonucleotides with anticancer properties according to claim 1, distinct in that the molecular charge of the nucleotide bases is changed to the opposite through the formation of a substitution amino group as a result of an alkylation reaction between monochloracetic acid and the amino groups of the nucleotide bases with the creation of new carboxyl groups.

4. A method of obtaining modified antisense oligonucleotides with anticancer properties, distinct in that to obtain them, first a partial hydrolysis of polynucleotides is conducted, and then a process of chemical modification of the mass (assembly) of oligonucleotides obtained with a change in their molecular charge is conducted; this is used as an anticancer vehicle for the composition of the antisense oligonucleotides obtained.

5. A method of obtaining modified antisense oligonucleotides with anticancer properties according to claim 4, distinct in that DNA is used in the capacity of polynucleotides.

6. A method of obtaining modified antisense oligonucleotides with anticancer properties according to claim 4, distinct in that RNA is used in the capacity of polynucleotides.

7. A method of obtaining modified antisense oligonucleotides with anticancer properties according to claim 5, distinct in that yeast is the source of the DNA.

8. A method of obtaining modified antisense oligonucleotides with anticancer properties according to claim 5, distinct in that raw animal material is the source of the DNA.

9. A method of obtaining modified antisense oligonucleotides with anticancer properties according to claim 5, distinct in that raw plant material is the source of the DNA.

10. A method of obtaining modified antisense oligonucleotides with anticancer properties according to claim 5, distinct in that raw microbial material is the source of the DNA.

11. A method of obtaining modified antisense oligonucleotides with anticancer properties according to claim 6, distinct in that yeast is the source of the RNA.

12. A method of obtaining modified antisense oligonucleotides with anticancer properties according to claim 6, distinct in that raw animal material is the source of the RNA.

13. A method of obtaining modified antisense oligonucleotides with anticancer properties according to claim 6, distinct in that raw plant material is the source of the RNA.

14. A method of obtaining modified antisense oligonucleotides with anticancer properties according to claim 6, distinct in that raw microbial material is the source of the RNA.

15. A method of obtaining modified antisense oligonucleotides with anticancer properties according to claim 4, distinct in that a mixture of RNA and DNA from yeast is used.

16. A method of obtaining modified antisense oligonucleotides with anticancer properties according to claim 4, distinct in that a mixture of RNA and DNA from raw animal material is used.

17. A method of obtaining modified antisense oligonucleotides with anticancer properties according to claim 4, distinct in that a mixture of RNA and DNA from raw plant material is used.

18. A method of obtaining modified antisense oligonucleotides with anticancer properties according to claim 4, distinct in that a mixture of RNA and DNA from raw microbial material is used.

19. A method of obtaining modified antisense oligonucleotides with anticancer properties according to claim 4, distinct in that for the partial hydrolysis of the polynucleotides, enzymatic hydrolysis is used.

20. A method of obtaining modified antisense oligonucleotides with anticancer properties according to claim 4, distinct in that for the partial hydrolysis of the polynucleotides, acid hydrolysis is used.

21. A method of obtaining modified antisense oligonucleotides with anticancer properties according to claim 4, distinct in that for the partial hydrolysis of the polynucleotides, alkaline hydrolysis is used.

22. A method of obtaining modified antisense oligonucleotides with anticancer properties according to claim 4, distinct in that for the partial hydrolysis of the polynucleotides, synthetic nucleases are used.

23. A method of obtaining modified antisense oligonucleotides with anticancer properties according to claim 4, distinct in that for the chemical modification of the oligonucleotide mass, succinic anhydride is used.

24. A method of obtaining modified antisense oligonucleotides with anticancer properties according to claim 4, distinct in that for the chemical modification of the oligonucleotide mass, monochloracetic acid is used.
